SEQUENCE LISTING (31 sequences)

SEQ ID NO: 1 - protein, 115 aa, Artificial Sequence
  Description of Artificial Sequence Synthetic polypeptide
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 2 - protein, 107 aa, Artificial Sequence
  Description of Artificial Sequence Synthetic polypeptide
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 3 - protein, 118 aa, Artificial Sequence
  Description of Artificial Sequence Synthetic polypeptide
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 4 - protein, 111 aa, Artificial Sequence
  Description of Artificial Sequence Synthetic polypeptide
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 5 - protein, 650 aa, Artificial Sequence
  Description of Artificial Sequence Synthetic polypeptide
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 657 aa, Artificial Sequence
  Description of Artificial Sequence Synthetic polypeptide
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 41 aa, Artificial Sequence
  Description of Artificial Sequence Synthetic polypeptide
RSKRSRLLHSDYMNMTPRRPGPTRKHQYPYAPPRDFAAYRS

SEQ ID NO: 8 - protein, 41 aa, Artificial Sequence
  Description of Artificial Sequence Synthetic polypeptide
RSKRSRGGHSDYMNMTPRRPGPTRKHYQPYAPPRDFAAYRS

SEQ ID NO: 9 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 10 - protein, 42 aa, Artificial Sequence
  Description of Artificial Sequence Synthetic polypeptide
ALYLLRRDQRLPPDAHKPPGGGSFRTPIQEEQADAHSTLAKI

SEQ ID NO: 11 - protein, 21 aa, Artificial Sequence
  Description of Artificial Sequence Synthetic peptide
LCYLLDGILFIYGVILTALFL

SEQ ID NO: 12 - protein, 27 aa, Artificial Sequence
  Description of Artificial Sequence Synthetic peptide
FWVLVVVGGVLACYSLLVTVAFIIFWV

SEQ ID NO: 13 - protein, 28 aa, Artificial Sequence
  Description of Artificial Sequence Synthetic peptide
MFWVLVVVGGVLACYSLLVTVAFIIFWV

SEQ ID NO: 14 - protein, 22 aa, Artificial Sequence
  Description of Artificial Sequence Synthetic peptide
MALIVLGGVAGLLLFIGLGIFF

SEQ ID NO: 15 - protein, 21 aa, Artificial Sequence
  Description of Artificial Sequence Synthetic peptide
IYIWAPLAGTCGVLLLSLVIT

SEQ ID NO: 16 - protein, 23 aa, Artificial Sequence
  Description of Artificial Sequence Synthetic peptide
IYIWAPLAGTCGVLLLSLVITLY

SEQ ID NO: 17 - protein, 24 aa, Artificial Sequence
  Description of Artificial Sequence Synthetic peptide
IYIWAPLAGTCGVLLLSLVITLYC

SEQ ID NO: 18 - protein, 24 aa, Artificial Sequence
  Description of Artificial Sequence Synthetic peptide
IISFFLALTSTALLFLLFFLTLRF

SEQ ID NO: 19 - protein, 3 aa, Artificial Sequence
  Description of Artificial Sequence Synthetic peptide
AAA

SEQ ID NO: 20 - protein, 10 aa, Artificial Sequence
  Description of Artificial Sequence Synthetic peptide
GGGSSGGGSG

SEQ ID NO: 21 - protein, 12 aa, Artificial Sequence
  Description of Artificial Sequence Synthetic peptide
ESKYGPPCPPCP

SEQ ID NO: 22 - protein, 12 aa, Artificial Sequence
  Description of Artificial Sequence Synthetic peptide
ESKYGPPCPSCP

SEQ ID NO: 23 - protein, 22 aa, Artificial Sequence
  Description of Artificial Sequence Synthetic peptide
ESKYGPPCPPCPGGGSSGGGSG

SEQ ID NO: 24 - protein, 39 aa, Artificial Sequence
  Description of Artificial Sequence Synthetic polypeptide
IEVMYPPPYLDNEKSNGTIIHVKGKHLCPSPLFPGPSKP

SEQ ID NO: 25 - protein, 48 aa, Artificial Sequence
  Description of Artificial Sequence Synthetic polypeptide
AKPTTTPAPRPPTPAPTIASQPLSLRPEACRPAAGGAVHTRGLDFACD

SEQ ID NO: 26 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 27 - protein, 129 aa, Artificial Sequence
  Description of Artificial Sequence Synthetic polypeptide
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 229 aa, Artificial Sequence
  Description of Artificial Sequence Synthetic polypeptide
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 229 aa, Artificial Sequence
  Description of Artificial Sequence Synthetic polypeptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 107 aa, Artificial Sequence
  Description of Artificial Sequence Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2020/0222462 A1
(43) Pub. Date: Jul. 16, 2020

(54) METHODS OF TREATMENT USING DECITABINE AND A CD123-TARGETED THERAPY

(71) Applicants: Mustang Bio, Inc., New York, NY (US); City of Hope, Duarte, CA (US)

(72) Inventors: Pam Cohen, New York, NY (US); Sadik Kassim, Milton, MA (US); Ekta Patel, New York, NY (US); Elizabeth Budde, Duarte, CA (US); Stephen J. Forman, Duarte, CA (US)

(73) Assignees: Mustang Bio, Inc., New York, NY (US); City of Hope, Duarte, CA (US)

(21) Appl. No.: 16/698,358

(22) Filed: Nov. 27, 2019

Related U.S. Application Data
(60) Provisional application No. 62/773,632, filed on Nov. 30, 2018.

(51) Int. Cl. A61K 35/17; C07K 14/705; C07K 16/28; A61P 35/00

(52) U.S. Cl. CPC A61K 35/17; A61P 35/00; C07K 16/2866; C07K 14/70503

(57) ABSTRACT

The present disclosure relates generally to methods treatment of hematological cancers, such as blastic plasmacytoid dendritic cell neoplasm (BPDCN), acute myeloid leukemia (AML), or Myelodysplastic Syndrome (MDS), comprising a combination of decitabine and a CD123-targeted therapy. In particular, the disclosed methods involve pretreatment of a patient with decitabine prior to administration of a CD123-targeted therapy.

### CROSS REFERENCE TO RELATED APPLICATION

This application claims priority under 35 U.S.C. § 119(e) to U.S. provisional application 62/773,632, filed Nov. 30, 2018, the entire contents of which are incorporated herein by reference.

### FIELD OF INVENTION

The present disclosure relates generally to methods treatment of hematological cancers, such as blastic plasmacytoid dendritic cell neoplasm (BPDCN), acute myeloid leukemia (AML), or Myelodysplastic Syndrome (MDS), comprising a combination of decitabine and a CD123-targeted therapy. In particular, the disclosed methods involve pretreatment of a patient with decitabine prior to administration of a CD123-targeted therapy.

### BACKGROUND

First line treatments for many hematological cancers, including blastic plasmacytoid dendritic cell neoplasm (BPDCN), acute myeloid leukemia (AML), or Myelodysplastic Syndrome (MDS), have remained largely unchanged for the last 50 years. Although standard induction chemotherapy can induce remissions in some cases, many patients eventually relapse and succumb to the disease. Therefore, the development of novel therapeutics for AML is crucial.

Targeted therapies that rely on binding or interaction with a particular marker on a cancerous cell have shown promise in the treatment of several cancers. CD123 (i.e., interleukin 3 receptor alpha chain or IL-3Rα) is one such target. CD123 is expressed on various malignancies including acute and chronic myeloid leukemia, hairy cell leukemia, B-cell lineage acute lymphoblastic leukemia, and blastic plasmacytoid dendritic cell neoplasms. Additionally, CD123 is not typically expressed on normal hematopoietic stem cells, thus making CD123 a seemingly attractive target for targeted therapies. However, despite several CD123-targeted therapies undergoing clinical trials, many CD123-targeted therapies have experienced far less clinical efficacy than one might expect. Accordingly, there is a need in the art to improve CD123 targeting when treating hematological cancers, and the present disclosure fulfils this need.

### SUMMARY

The present disclosure provides methods treating hematological cancers, such as blastic plasmacytoid dendritic cell neoplasm (BPDCN), acute myeloid leukemia (AML), or Myelodysplastic Syndrome (MDS), with a combination of decitabine and a CD123-targeted therapy.

In one aspect, the disclosure relates to methods of treating a hematological cancer comprising, administering an effective amount of a cytidine analog to an individual with a hematological cancer and subsequently administering to the individual a therapeutic agent that targets CD123.

In some embodiments, the cytidine analog is selected from the group consisting of decitabine, guadecitabine, and 5-azacytidine.

In some embodiments, the hematological cancer is blastic plasmacytoid dendritic cell neoplasm (BPDCN), acute myeloid leukemia (AML), or Myelodysplastic Syndrome (MDS). In some embodiments, the hematological cancer is characterized by cancerous cells that overexpress CD123.

In some embodiments, the therapeutic agent that targets CD123 is a T-cell or a natural killer (NK) cell expressing a chimeric antigen receptor (CAR) that binds to CD123 (i.e., a CD123-specific CAR), while in some embodiments, the therapeutic agent that targets CD123 is selected from the group consisting of a monoclonal antibody (e.g., talacotuzumab), a bispecific antibody (e.g., flotetuzumab, XmAb14045, JNJ-63709178, APVO436, or APVO437), an antibody-drug conjugate (e.g., SGN-CD123A or IMGN632), and an immunotoxin-peptide conjugate (e.g., SL-401).

In another aspect, the disclosure relates to methods of conditioning an individual with blastic plasmacytoid dendritic cell neoplasm (BPDCN), acute myeloid leukemia (AML), or Myelodysplastic Syndrome (MDS) for treatment with T-cells expressing a chimeric antigen receptor (CAR) that binds to CD123 comprising, administering to the individual with BPDCN, AML, or MDS an effective dose of decitabine, thereby increasing expression of CD123 on BPDCN, AML or MDS stem cells and/or blast; and administering to the individual a population of T-cells expressing a CAR that binds to CD123.

In yet another embodiment, the disclosure relates to methods of conditioning an individual with blastic plasmacytoid dendritic cell neoplasm (BPDCN), acute myeloid leukemia (AML), or Myelodysplastic Syndrome (MDS) for treatment with T-cells expressing a chimeric antigen receptor (CAR) that binds to CD123 comprising, administering to the individual an effective dose of decitabine at least about a week prior to administration of a CAR, thereby increasing expression of CD123 on BPDCN, AML, or MDS stem cells and/or blast prior to administration of a CAR; and administering to the individual a population of T-cells expressing a CAR that binds to CD123.

In some embodiments of any of the foregoing aspect, the CAR comprises (i) the complementarity determining regions (CDRs) of the heavy chain variable region disclosed in SEQ ID NO:1 and the CDRS of the light chain variable region disclosed in SEQ ID NO:2; or (ii) the CDRs of the heavy chain variable region disclosed in SEQ ID NO:3 and the CDRs of the light chain variable region disclosed in SEQ ID NO:4. For example, in some embodiments, the CAR comprises (i) a heavy chain variable region comprising SEQ ID NO:1 and a light chain variable region comprising SEQ ID NO:2; or (ii) a heavy chain variable region comprising SEQ ID NO:3 and a light chain variable region comprising SEQ ID NO:4.

In some embodiments of any of the foregoing aspect, the CAR comprises a CD28 costimulatory domain, a 4-1BB costimulatory domain, or a combination thereof.

In some embodiments of any of the foregoing aspect, the CAR comprises a CD28 transmembrane domain, a CD4 transmembrane domain, or a CD8 transmembrane domain.

In some embodiments of any of the foregoing aspect, the CAR comprises a hinge domain derived from an IgG4 Fc region or an IgG2 Fc region.

In some embodiments of any of the foregoing aspect, the CAR comprises a CD123 binding domain, a CD28 costimulatory domain, a CD28 transmembrane domain, a hinge derived from an IgG4 Fc region, and a CD3ζ domain.

In some embodiments of any of the foregoing aspect, the CAR comprises SEQ ID NO:5 or SEQ ID NO:6.

In some embodiments, the population of T-cells expressing a CAR that binds to CD123 is administered at a dose of 1.0×10⁴-12.0×10⁶ cells/kg or any value in between, while in some embodiments, the population of T-cells expressing a CAR that binds to CD123 is administered at a dose of 25×10⁴-750×10⁶ cells or any value in between.

In some embodiments of any of the foregoing aspect, the CAR is bispecific for CD123 and a different antigenic target (e.g., CD33, CD19, CD20, HER2, CS-1, PSCA, IL-13R, etc.). For example, in some embodiments, the bispecific CAR comprises a CD123 binding domain comprising (i) a heavy chain variable region comprising SEQ ID NO:1 and a light chain variable region comprising SEQ ID NO:2; or (ii) a heavy chain variable region comprising SEQ ID NO:3 and a light chain variable region comprising SEQ ID NO:4; and a second binding domain specific for a different antigenic target.

In some embodiments of any of the foregoing aspect, the decitabine or another cytidine analog is administered at a dose of about 20 mg/m² per day. In some embodiments, the decitabine or another cytidine analog is administered at least a week prior to commencing treatment with the CD123-targeted therapy or CD123-specific CAR. In some embodiments, the decitabine or another cytidine analog is administered for 3-5 days during the week prior to commencing treatment with the CD123-targeted therapy or CD123-specific CAR.

In some embodiments of the disclosed methods, the individual has BPDCN, while in others the individual has AML, while in other still the individual has MDS.

In some embodiments, the patient continues to receive decitabine or another cytidine analog after initiation of CD123-targeting therapy for at least an additional 1-2 weeks. In some embodiments, the patient continues to receive decitabine or another cytidine analog after initiation of CD123-targeting therapy for at least the duration of the CD123-targeting therapy.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF DRAWINGS

FIG. 1 shows live cell counts for AML cell lines and Normal donor CD34+ Bone marrow cells post decitabine, 5-azacytidine and guadecitabine treatment: (a) Kasumi-1, SKM-1 and MOLM-13 (n=3) and (b) normal donor CD34+ bone marrow cells (n=4) were serially dosed with indicated drugs and subjected to flow cytometry analysis on day 3 and 7 post treatment. Number of live cells was determined by excluding DAPI+ dead cells in each well. Average of triplicates is presented on the graph.

FIG. 2 shows CD123 expression on AML cell lines and Normal donor CD34+ Bone marrow cells post decitabine, 5-azacytidine and guadecitabine treatment: (a) Kasumi-1, SKM-1 and MOLM-13 (n=3) and (b) Normal donor CD34+ bone marrow cells (n=4) were serially dosed with indicated drugs and subjected to flow cytometry analysis on Day 3 and 7 post treatment. (c) Fold change in CD123 MFI at 1000 nM clinically relevant decitabine dose (P values are calculated by student Test). Cells are gated on live cells. Blast cells in AML cell lines were defined as CD33 or CD34 positive. Primary HSCs were defined as CD45^{dim}SSC^{low}CD34^{high}CD38− and multipotent progenitors were defined as CD34^{high}CD38+, data represents mean of four normal donors and the error bars represent Standard deviation. Fold change in CD123 Mean Fluorescence Intensity (MFI) with respect to DMSO treated cells is presented.

FIG. 3 shows PD-L 1 expression on AML cell lines and Normal donor CD34+ Bone marrow cells post decitabine, 5-Azacytidine and guadecitabine treatment: (a) Kasumi-1, SKM-1 and MOLM-13 (n=3) and (b) Normal donor CD34+ bone marrow cells (n=4) were serially dosed with indicated drugs and subjected to flow cytometry analysis on day 3 and 7 post treatment. Cells are gated on live cells. Blast cells in AML cell lines were defined as CD33 or CD34 positive. Primary HSCs were defined as CD45^{dim}SSC^{low}CD34^{high}CD38− and multipotent progenitors were defined as CD34^{high}CD38+, data represents mean of four normal donors and the error bars represent Standard deviation. Fold change in PD-L 1 Mean Fluorescence Intensity (MFI) with respect to DMSO treated cells is presented.

### DETAILED DESCRIPTION

The compositions and methods of the present disclosure employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means the recited quantity exactly and plus or minus 10%. For example, “about 10” should be understood to mean “10” and “9-11”.

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the term an “isolated antibody” is intended to refer to an antibody which is substantially free of other antibodies having different antigenic specificities (e.g., an isolated antibody that specifically binds to CD123 and is substantially free of antibodies that do not bind to CD123). An isolated antibody that specifically binds to an epitope of CD123 may, however, have cross-reactivity to other proteins. However, the antibody preferably always binds to human CD123. In addition, an isolated antibody is typically substantially free of other cellular material and/or chemicals.

As used herein, the term “humanized antibody” refers to an antibody that comprises the CDRs of antibodies derived from mammals other than human, and the framework (FR) region and the constant region of a human antibody. A humanized antibody is useful as an effective component in a therapeutic agent according to the present disclosure since antigenicity of the humanized antibody in human body is lowered.

As used herein, the term “recombinant human antibody” includes all human antibodies that are prepared, expressed, created or isolated by recombinant means, including but not limited to (a) antibodies isolated from an animal (e.g., a mouse) that is transgenic or transchromosomal for human immunoglobulin genes or a hybridoma prepared therefrom, (b) antibodies isolated from a host cell transformed to express the antibody (e.g., from a transfectoma), (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, the term “glycosylation pattern” is defined as the pattern of carbohydrate units that are covalently attached to a protein, more specifically to an immunoglobulin protein.

As used herein, the phrases “therapeutically effective amount” and “therapeutic level” mean that drug dosage or plasma concentration in a subject, respectively, that provides the specific pharmacological effect for which the drug is administered in a subject in need of such treatment, i.e. to reduce, ameliorate, or eliminate the symptoms or effects of a hematological cancer, malignant disease, or cancer cell proliferation. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the cancer, malignant disease, or cancer cell proliferation, among other factors.

The terms “treatment” or “treating” as used herein with reference to cancer, malignant disease, or cancer cell proliferation refer to reducing, ameliorating or eliminating one or more symptoms or effects of cancer, malignant disease, or cancer cell proliferation.

As used herein, the term “effective amount” when used in relation to decitabine or another cytidine analog means an amount that is sufficient to upregulate expression of CD123. Upregulation can be determined by conventional means known in the art, including but not limited to Q-PCR, RT-PCR, flow cytometry, Western blotting, RNAseq, etc.

### II. CD123-Targeted Therapies

Provided herein are CD123-targeted therapies that may be used, among other reasons, to treat hematological cancer. The CD123-targeted therapies of the present disclosure share the common trait of specifically binding to or directly interacting with CD123. For example, for the purposes of the present disclosure, a CD123-targeted therapy may comprise a T-cell or a natural killer (NK) cell expressing a chimeric antigen receptor (CAR) that binds to CD123, an anti-CD123 monoclonal antibody or bispecific antibody, an antibody-drug conjugate, or an immunotoxin-peptide conjugate that binds to CD123.

In some embodiments, the CD123-targeted therapy may be a T-cell or a natural killer (NK) cell that expresses a chimeric antigen receptor (CAR) comprising a binding domain that binds specifically to CD123. A CD123-specific CAR may comprise the complementarity determining regions or heavy and light chain variable regions of known anti-CD123 antibodies or other ligands that bind to CD123 (e.g., IL-3 or a CD123-binding fragment thereof). Exemplary variable sequences that can be incorporated into CD123-specific CARs for the purposes of the disclosed methods are provided in Table 1 below, but these examples should not be construed as limiting.

TABLE 1
Exemplary Anti-CD123 Binding Domains and CARs

| Seq ID | | |
|---|---|---|
| No: | Description | Sequence |
| 1 | Variable heavy chain | QVQLQQPGAELVRPGASVKLSCKASGYTFTSYWMNWVKQRPD |
| | of Ab 26292 | QGLEWIGRIDPYDSETHYNQKFKDKAILTVDKSSSTAYMQLSSL |
| | | TSEDSAVYYCARGNWDDYWGQGTTLTVSS |
| 2 | Variable light chain | DVQITQSPSYLAASPGETITINCRASKSISKDLAWYQEKPGKTNK |
| | of Ab 26292 | LLIYSGSTLQSGIPSRFSGSGSGTDFTLTISSLEPEDFAMYYCQQH |
| | | NKYPYTFGGGTKLEIK |
| 3 | Variable heavy chain | QIQLVQSGPELKKPGETVKISCKASGYIFTNYGMNWVKQAPGKS |
| | of Ab 32716 | FKWMGWINTYTGESTYSADFKGRFAFSLETSASTAYLHINDLKN |
| | | EDTATYFCARSGGYDPMDYWGQGTSVTVSS |
| 4 | Variable light chain | DIVLTQSPASLAVSLGQRATISCRASESVDNYGNTFMHWYQQKP |
| | of Ab 32716 | GQPPKLLIYRASNLESGIPARFSGSGSRTDFTLTINPVEADDVATY |
| | | YCQQSNEDPPTFGAGTKLELK |
| 5 | Exemplary 26292 | QVQLQQPGAELVRPGASVKLSCKASGYTFTSYWMNWVKQRPD |
| | CAR | QGLEWIGRIDPYDSETHYNQKFKDKAILTVDKSSSTAYMQLSSL |
| | | TSEDSAVYYCARGNWDDYWGQGTTLTVSSGGGGSGGGGSGGG |
| | | GSDVQITQSPSYLAASPGETITINCRASKSISKDLAWYQEKPGKT |
| | | NKLLIYSGSTLQSGIPSRFSGSGSGTDFTLTISSLEPEDFAMYYCQ |
| | | QHNKYPYTFGGGTKLEIKESKYGPPCPPCPAPEFEGGPSVFLFPP |
| | | KPKDTLMISRTPEVTCVVVDVSQEDPEVQFNWYVDGVEVHNAK |
| | | TKPREEQFQSTYRVVSVLTVLHQDWLNGKEYKCKVSNKGLPSSI |
| | | EKTISKAKGQPREPQVYTLPPSQEEMTKNQVSLTCLVKGFYPSDI |
| | | AVEWESNGQPENNYKTTPPVLDSDGSFFLYSRLTVDKSRWQEG |
| | | NVFSCSVMHEALHNHYTQKSLSLSLGKMFWVLVVVGGVLACY |
| | | SLLVTVAFIIFWVRSKRSRGGHSDYMNMTPRRPGPTRKHYQPYA |
| | | PPRDFAAYRSGGGRVKFSRSADAPAYQQGQNQLYNELNLGRRE |
| | | EYDVLDKRRGRDPEMGGKPRRKNPQEGLYNELQKDKMAEAYS |
| | | EIGMKGERRRGKGHDGLYQGLSTATKDTYDALHMQALPPR |
| 6 | Exemplary 32716 | QIQLVQSGPELKKPGETVKISCKASGYIFTNYGMNWVKQAPGKS |
| | CAR | FKWMGWINTYTGESTYSADFKGRFAFSLETSASTAYLHINDLKN |
| | | EDTATYFCARSGGYDPMDYWGQGTSVTVSSGGGGSGGGGSGG |
| | | GGSDIVLTQSPASLAVSLGQRATISCRASESVDNYGNTFMHWYQ |
| | | QKPGQPPKLLIYRASNLESGIPARFSGSGSRTDFTLTINPVEADDV |
| | | ATYYCQQSNEDPPTFGAGTKLELKESKYGPPCPPCPAPEFEGGPS |
| | | VFLFPPKPKDTLMISRTPEVTCVVVDVSQEDPEVQFNWYVDGVE |
| | | VHNAKTKPREEQFQSTYRVVSVLTVLHQDWLNGKEYKCKVSN |
| | | KGLPSSIEKTISKAKGQPREPQVYTLPPSQEEMTKNQVSLTCLVK |
| | | GFYPSDIAVEWESNGQPENNYKTTPPVLDSDGSFFLYSRLTVDK |
| | | SRWQEGNVFSCSVMHEALHNHYTQKSLSLSLGKMFWVLVVVG |
| | | GVLACYSLLVTVAFIIFWVRSKRSRGGHSDYMNMTPRRPGPTRK |
| | | HYQPYAPPRDFAAYRSGGGRVKFSRSADAPAYQQGQNQLYNEL |
| | | NLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |

In some embodiments, the CD123-specifc CAR comprises at least one costimulatory domain (e.g., a costimulatory region of CD28, CD28gg, 4-1BB, CD3, CD27, ICOS, OX40, HVEM, CD30 and/or any other member of the family of T cell co-stimulatory molecules), a transmembrane domain (e.g., a transmembrane portion of CD28, CD4, CD8, 4-1BB, CD27, ICOS, OX40, HVEM, or CD30), a spacer and/or hinge (an IgG4 hinge or derivative thereof, an IgG2 hinge or derivative thereof, a CD28 hinge, or a CD8 hinge), and an antigen-binding domain, such as a scFv. Exemplary costimulatory domains, transmembrane domains, and spacers are provided in the following tables.

TABLE 2
Exemplary Costimulatory Domains

| SEQ ID | | |
|---|---|---|
| NO: | Description | Sequence |
| 7 | CD28 | RSKRSRLLHSDYMNMTPRRPGPTRKHQYPYAPPRDFAAYRS |
| 8 | CD28gg | RSKRSRGGHSDYMNMTPRRPGPTRKHYQPYAPPRDFAAYRS |
| 9 | 4-1BB | KRGRKKLLYIFKOPFMRPVOTTOEEDGCSCRFPEEEEGGCEL |
| 10 | OX40 | ALYLLRRDORLPPDAHKPPGGGSFRTPIQEEQADAHSTLAKI |

TABLE 3
Exemplary Transmembrane Domains
SEQ

| ID NO: | Description | Sequence |
|---|---|---|
| 11 | CD3z | LCYLLDGILFIYGVILTALFL |
| 12 | CD28 | FWVLVVVGGVLACYSLLVTVAFIIFWV |
| 13 | CD28(M) | MFWVLVVVGGVLACYSLLVTVAFIIFWV |
| 14 | CD4 | MALIVLGGVAGLLLFIGLGIFF |
| 15 | CD8(i) | IYIWAPLAGTCGVLLLSLVIT |
| 16 | CD8(ii) | IYIWAPLAGTCGVLLLSLVITLY |
| 17 | CD8(iii) | IYIWAPLAGTCGVLLLSLVITLYC |
| 18 | 4-1BB | IISFFLALTSTALLFLLFFLTLRF |

TABLE 4
Exemplary Linkers and Hinges

| SEQ | | |
|---|---|---|
| ID NO: | Description | Sequence |
| 19 | A3 | AAA |
| 20 | Linker | GGGSSGGGSG |
| 21 | IgG4 hinge (S228P) | ESKYGPPCPPCP |
| 22 | IgG4 hinge | ESKYGPPCPSCP |
| 23 | IgG4 hinge + linker | ESKYGPPCPPCPGGGSSGGGSG |
| 24 | CD28 hinge | IEVMYPPPYLDNEKSNGTIIHVKGKHLCPSPLFPGPSKP |
| 25 | CD8 hinge (48 AA) | AKPTTTPAPRPPTPAPTIASOPLSLRPEACRPAAGGAVHTRGLDF |
| | | ACD |
| 26 | CD8 hinge (45 AA) | TTTPAPRPPTPAPTIASOPLSLRPEACRPAAGGAVHTRGLDFACD |
| 27 | IgG4 (HL-CH3) | ESKYGPPCPPCPGGGSSGGGSGGQPREPQVYTLPPSQEEMTKNQ |
| | | VSLTCLVKGFYPSDIAVEWESNGQPENNYKTTPPVLDSDGSFFL |
| | | YSRLTVDKSRWQEGNVFSCSVMHEALHNHYTOKSLSLSLGK |
| 28 | IgG4 (L235E, | ESKYGPPCPSCPAPEFEGGPSVFLFPPKPKDTLMISRTPEVTCVVV |
| | N297Q) | DVSQEDPEVOFNWYVDGVEVHOAKTKPREEQFNSTYRVVSVLT |
| | | VLHODWLNGKEYKCKVSNKGLPSSIEKTISKAKGQPREPOVYTL |
| | | PPSOEEMTKNOVSLTCLVKGFYPSDIAVEWESNGOPENNYKTTP |
| | | PVLDSDGSFFLYSRLTVDKSRWQEGNVFSCSVMHEALHNHYTO |
| | | KSLSLSLGK |
| 29 | IgG4 (S228P, | ESKYGPPCPPCPAPEFEGGPSVFLFPPKPKDTLMISRTPEVTCVVV |
| | L235E, N297Q) | DVSQEDPEVOFNWYVDGVEVHOAKTKPREEOFNSTYRVSVLTV |
| | | LHODWLNGKEYKCKVSNKGLPSSIEKTISKAKGOPREPOVYTLP |
| | | PSOEEMTKNOVSLTCLVKGFYPSDIAVEWESNGOPENNYKTTPP |
| | | VLDSDGSFFLYSRLTVDKSRWOEGNVFSCSVMHEALHNHYTOK |
| | | SLSLSLGK |
| 30 | IgG4 (CH3) | GQPREPOVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTOKSLSLSLGK |

In some embodiments, the CD123-specific CAR comprises a CD3ζ signaling domain (RVKFSRSADAPAYQQGQNQLYNELNLGRREEYDVLDKRRGRDPEMGGKPRRKNPQE GLYNELQKDKMAEAYSEIGMKGERRRGKGHDGLYOGLSTATKDTYDALHMOALPPR; SEQ ID NO:31).

In some embodiments of the disclosed methods, the CD123-specific CAR may comprise the CDRS or variable regions of antibodies 26292 or 32716 (e.g., SEQ ID NOs: 1/2 or 3/4), an IgG4 hinge that may optionally comprise substitution mutations S228P, L235E, and/or N297Q (e.g., SEQ ID NOs: 27-30), a CD8 transmembrane domain (e.g., SEQ ID NOs: 15-17), a 4-1BB costimulatory domain (e.g., SEQ ID NO:9), and a CD3ζ signaling domain (e.g., SEQ ID NO:31). In some embodiments of the disclosed methods, the CD123-specific CAR may comprise the CDRS or variable regions of antibodies 26292 or 32716 (e.g., SEQ ID NOs: 1/2 or 3/4), an IgG4 hinge that may optionally comprise substitution mutations S228P, L235E, and/or N297Q (e.g., SEQ ID NOs: 27-30), a CD28 transmembrane domain (e.g., SEQ ID NOs: 12 or 13), a CD28 costimulatory domain (e.g., SEQ ID NOs: 7 or 8), and a CD3ζ signaling domain (e.g., SEQ ID NO:31). In some embodiments, the CD123-specific CAR may comprise more than one costimulatory domain, for example, a 4-1BB costimulatory domain and a CD28 costimulatory domain (e.g., SEQ ID NOs: 7 or 8), a 4-1BB costimulatory domain and an OX40 costimulatory domain, a CD28 costimulatory domain (e.g., SEQ ID NOs: 7 or 8) and an OX40 costimulatory domain.

In some embodiments of the disclosed methods, the CD123 CAR may be bispecific (e.g., the bispecific CARs disclosed in U.S. 2018/0162939), comprising a CD123-specific binding domain and a binding domain specific for another antigen, such as CD33 (e.g., the CDRs or variable regions of gemtuzumab), HER2 (e.g., the CDRs or variable domains of 4D5 as disclosed in PCT/US2016/060724), IL-13R (e.g., an E13Y binding domain as disclosed in PCT/US2015/051089), CS-1 (e.g., the CDRs or variable domains of CS1R as disclosed in PCT/US2015/064303), PSCA (e.g., the CDRs or variable domains of A11 as disclosed in PCT/US2008/075291 or PCT/US2016/055761), CD20 (e.g., the CDRs or variable domains of 1.5.3 as disclosed in PCT/US2017/023098), or CD19 (e.g., the CDRs or variable domains of FMC63 as disclosed in PCT/US2014/028961).

In some embodiments, the CD123-targeted therapy comprises an anti-CD123 monoclonal antibody. The antibody by be chimeric, human, or humanized, and may comprise, for example, the CDRs or the variable domains of antibodies 26292 or 32716 (e.g., SEQ ID NOs: 1/2 or 3/4) shown in Table 1 above. Other known anti-CD123 monoclonal antibodies that may be used in the disclosed methods including, but are not limited to, talacotuzumab.

In some embodiments, the CD123-targeted therapy comprises a bispecific antibody that binds to CD123 and another antigen. Exemplary bispecific antibodies that may be used in the disclosed methods include, but are not limited to, flotetuzumab, XmAb14045, JNJ-63709178, APV0436, and APV0437. Additional bispecific antibodies include those that bind to CD123 and CD33 (e.g., the CDRs or variable regions of gemtuzumab), HER2 (e.g., the CDRs or variable domains of 4D5 as disclosed in PCT/US2016/060724), IL-13R (e.g., an E13Y binding domain as disclosed in PCT/US2015/051089), CS-1 (e.g., the CDRs or variable domains of CS1R as disclosed in PCT/US2015/064303), PSCA (e.g., the CDRs or variable domains of A11 as disclosed in PCT/US2008/075291 or PCT/US2016/055761), CD20 (e.g., the CDRs or variable domains of 1.5.3 as disclosed in PCT/US2017/023098), or CD19 (e.g., the CDRs or variable domains of FMC63 as disclosed in PCT/US2014/028961).

In some embodiments, the CD123-targeted therapy comprises an antibody-drug conjugate (ADC) in which an anti-CD123 antibody or bispecific antibody is conjugated to a therapeutic moiety, such as a chemotherapeutic drug. Exemplary ADCs that may be used in the disclosed methods include, but are not limited to, SGN-CD123A and IMGN632.

In some embodiments, the CD123-targeted therapy comprises an immunotoxin-peptide conjugate, in which a peptide that is capable of binding to CD123 is conjugated to an immunotoxin. An exemplary immunotoxin-peptide conjugate that may be used in the disclosed methods includes, but are not limited to, SL-401 (a diphtheria toxin-IL3 fusion protein which directly targets CD123+ cells).

A therapeutically effective amount of a CD123-targeted therapy administered to the patient may vary depending on the therapy being used, the size and age of the patient, and the disease being treated. For example, in the embodiments in which the CD123-targeted therapy is a T-cell or a natural killer (NK) cell expressing a CAR that binds to CD123, the therapeutically effective dose will generally be between 1.0×10⁴ cells/kg and 12.0×10⁶ cells/kg. Indeed, the T-cells expressing the CD123-specific CAR may be administered in a dose of about 1.0×10⁴ cells/kg, about 1.5×10⁴ cells/kg, about 2.0×10⁴ cells/kg, about 2.5×10⁴ cells/kg, about 3.0×10⁴ cells/kg, about 3.5×10⁴ cells/kg, about 4.0×10⁴ cells/kg, about 4.5×10⁴ cells/kg, about 5.0×10⁴ cells/kg, about 5.5×10⁴ cells/kg, about 6.0×10⁴ cells/kg, about 6.5×10⁴ cells/kg, about 7.0×10⁴ cells/kg, about 7.5×10⁴ cells/kg, about 8.0×10⁴ cells/kg, about 8.5×10⁴ cells/kg, about 9.0×10⁴ cells/kg, about 9.5×10⁴ cells/kg, about 1.0×10⁵ cells/kg, about 1.5×10⁵ cells/kg, about 2.0×10⁵ cells/kg, about 2.5×10⁵ cells/kg, about 3.0×10⁵ cells/kg, about 3.5×10⁵ cells/kg, about 4.0×10⁵ cells/kg, about 4.5×10⁵ cells/kg, about 5.0×10⁵ cells/kg, about 5.5×10⁵ cells/kg, about 6.0×10⁵ cells/kg, about 6.5×10⁵ cells/kg, about 7.0×10⁵ cells/kg, about 7.5×10⁵ cells/kg, about 8.0×10⁵ cells/kg, about 8.5×10⁵ cells/kg, about 9.0×10⁵ cells/kg, about 9.5×10⁵ cells/kg, about 1.0×10⁶ cells/kg, about 1.2×10⁶ cells/kg, about 1.4×10⁶ cells/kg, about 1.6×10⁶ cells/kg, about 1.8×10⁶ cells/kg, about 2.0×10⁶ cells/kg, about 2.2×10⁶ cells/kg, about 2.4×10⁶ cells/kg, about 2.6×10⁶ cells/kg, about 2.8×10⁶ cells/kg, about 3.0×10⁶ cells/kg, about 3.2×10⁶ cells/kg, about 3.4×10⁶ cells/kg, about 3.6×10⁶ cells/kg, about 3.8×10⁶ cells/kg, about 4.0×10⁶ cells/kg, about 4.2×10⁶ cells/kg, about 4.4×10⁶ cells/kg, about 4.6×10⁶ cells/kg, about 4.8×10⁶ cells/kg, about 5.0×10⁶ cells/kg, about 5.2×10⁶ cells/kg, about 5.4×10⁶ cells/kg, about 5.6×10⁶ cells/kg, about 5.8×10⁶ cells/kg, about 6.0×10⁶ cells/kg, about 6.2×10⁶ cells/kg, about 6.4×10⁶ cells/kg, about 6.6×10⁶ cells/kg, about 6.8×10⁶ cells/kg, about 7.0×10⁶ cells/kg, about 7.2×10⁶ cells/kg, about 7.4×10⁶ cells/kg, about 7.6×10⁶ cells/kg, about 7.8×10⁶ cells/kg, about 8.0×10⁶ cells/kg, about 8.2×10⁶ cells/kg, about 8.4×10⁶ cells/kg, about 8.6×10⁶ cells/kg, about 8.8×10⁶ cells/kg, about 9.0×10⁶ cells/kg, about 9.2×10⁶ cells/kg, about 9.4×10⁶ cells/kg, about 9.6×10⁶ cells/kg, about 9.8×10⁶ cells/kg, about 10.0×10⁶ cells/kg. about 10.2×10⁶ cells/kg, about 10.4×10⁶ cells/kg, about 10.6×10⁶ cells/kg, about 10.8×10⁶ cells/kg, about 11.0×10⁶ cells/kg. about 11.2×10⁶ cells/kg, about 11.4×10⁶ cells/kg, about 11.6×10⁶ cells/kg, about 11.8×10⁶ cells/kg, or about 12.0×10⁶ cells/kg. In some embodiments, the patient may be administered 1.5-11.5×10⁶ cells/kg, 2.0-11×10⁶ cells/kg, 2.5-10.5×10⁶ cells/kg, 3.0-10.0×10⁶ cells/kg, 3.5-9.5×10⁶ cells/kg, 4.0-9.0×10⁶ cells/kg, 4.5-8.5×10⁶ cells/kg, 5.0-8.0×10⁶ cells/kg, 5.5-7.5×10⁶ cells/kg, or 6.0-7.0×10⁶ cells/kg.

As an alternative dose calculation, in the embodiments in which the CD123-targeted therapy is a T-cell expressing a CAR that binds to CD123, the therapeutically effective dose will generally be between 25×10⁴-750×10⁶ cells. For example, the patient may be administered about 25×10⁴, about 50×10⁴, about 75×10⁴, about 100×10⁴, about 125×10⁴, about 150×10⁴, about 175×10⁴, about 200×10⁴, about 225×10⁴, about 250×10⁴, about 275×10⁴, about 300×10⁴, about 325×10⁴, about 350×10⁴, about 375×10⁴, about 400×10⁴, about 425×10⁴, about 450×10⁴, about 475×10⁴, about 500×10⁴, about 525×10⁴, about 550×10⁴, about 575×10⁴, about 600×10⁴, about 625×10⁴, about 650×10⁴, about 675×10⁴, about 700×10⁴, about 725×10⁴, about 750×10⁴, about 800×750×10⁴, about 825×10⁴, about 850×10⁴, about 875×10⁴, about 900×10⁴, about 925×10⁴, about 950×10⁴, about 975×10⁴, about 1000×10⁴, about 25×10⁵, about 50×10⁵, about 75×10⁵, about 100×10⁵, about 125×10⁵, about 150×10⁵, about 175×10⁵, about 200×10⁵, about 225×10⁵, about 250×10⁵, about 275×10⁵, about 300×10⁵, about 325×10⁵, about 350×10⁵, about 375×10⁵, about 400×10⁵, about 425×10⁵, about 450×10⁵, about 475×10⁵, about 500×10⁵, about 525×10⁵, about 550×10⁵, about 575×10⁵, about 600×10⁵, about 625×10⁵, about 650×10⁵, about 675×10⁵, about 700×10⁵, about 725×10⁵, about 750×10⁵, about 800×750×10⁵, about 825×10⁵, about 850×10⁵, about 875×10⁵, about 900×10⁵, about 925×10⁵, about 950×10⁵, about 975×10⁵, about 1000×10⁵, about 25×10⁶, about 50×10⁶, about 75×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, about 600×10⁶, about 625×10⁶, about 650×10⁶, about 675×10⁶, about 700×10⁶, about 725×10⁶, or about 750×10⁶ CD123-specific CAR T-cells.

In the embodiments in which the CD123-targeted therapy is a monoclonal or bispecific antibody, the therapeutically effective dose is generally from about 50 to about 1000 mg/kg, about 150 mg/kg to about 850 mg/kg, about 250 mg/kg to about 750 mg/kg, about 350 mg/kg to about 650 mg/kg, or about 450 mg/kg to about 550 mg/kg. In some embodiments, the therapeutically effective dose of an anti-CD123 monoclonal or bispecific antibody is from 50 to 1000 mg/kg, 150 mg/kg to 850 mg/kg, 250 mg/kg to 750 mg/kg, 350 mg/kg to 650 mg/kg, or 450 mg/kg to 550 mg/kg. In some embodiments, the therapeutically effective dose of an anti-CD123 monoclonal or bispecific antibody is a dose of about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600, about 650 mg/kg, about 700 mg/kg, about 750 mg/kg, about 800 mg/kg, about 850 mg/kg, about 900 mg/kg, about 950 mg/kg, or about 1000 mg/kg. In some embodiments, the therapeutically effective dose of an anti-CD123 monoclonal or bispecific antibody is a dose of about 3000 mg, about 3500 mg, about 4000 mg, about 4500 mg, about 5000 mg, about 5500 mg, about 6000, about 6500 mg, about 7000 mg, about 7500 mg, about 8000 mg, about 8500 mg, about 9000 mg, about 9500 mg, about 10000 mg, about 10500 mg, about 11000 mg, about 11500 mg, or about 12000 mg. When other antibody-related constructs are used, such as antibody fragments, they can be used at comparable doses adjusted for their different molecular weights and/or binding affinities.

The disclosed CD123-targeted therapies can be formulated in a pharmaceutical composition suitable for administration to a subject with a hematological cancer by any intended route of administration, as discussed in more detail below.

### III. Cytidine Analogs

Cytidine is a nucleoside that is forms when cytosine is attached to a ribose ring via a β-N1-glycosidic bond. Analogs of cytidine have historically been used as nucleic acid synthesis inhibitors for the treatment of various types of hematological and malignant diseases, such as myelodysplastic syndromes and acute myeloid leukemia.

It has now been discovered that cytidine analogs can upregulate CD123 in cancerous cells when it is administered to a patient. Similarly, CD123 expression in AML cells was quantitated by fluorescence-activated cell sorting, and a significant increase in CD123 expression was detected with the 3 and 5 day treatments of decitabine (p<0.001) and with the 5 day treatment for azacitidine (p<0.05). Addition of decitabine to the conditioning regimen of patients with AML or MDS may increase the chance that CD123-targeted CAR T cells will recognize their leukemic blast cells Presumably, other cytidine analogs, including but not limited to guadecitabine, could produce a similar upregulation of CD123 when administered in an effective amount. Accordingly, for the purposes of the disclosed methods, a patient with a hematological cancer can be pre-treated with a cytidine analog (e.g., decitabine, guadecitabine, or 5-azacytidine) prior to administration of a CD123-targeted therapy in order to increase the effectiveness of the CD123-targeted therapy by upregulated the expression of CD123 on the surface of the patient's cancer cells.

The structures of cytidine, decitabine, 5-azacytidine are shown in the formulas below to illustrate the structural similarities of these compounds, all of which share specific functional properties (e.g., they are nucleosides or nucleoside analogs and hypomethylating agents). Indeed, while not being bound by theory, it is believed that any hypomethylating agent that upregulates or increases expression of CD123 will be suitable for use in the pre-treatment conditioning regimens of the disclosed methods.

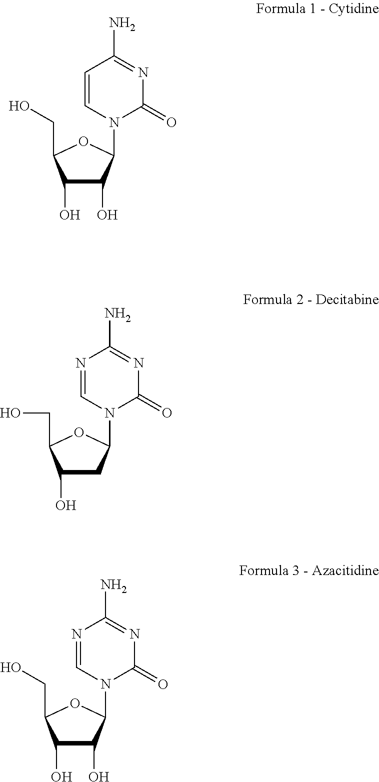

In some embodiments of the disclosed method, a patient with a hematological cancer (e.g., BPDCN, AML, or MDS) is administered an effective amount of a cytidine analog prior to being administered a CD123-targeted therapy. In some embodiments, the cytidine analog is decitabine, while in some embodiments, the cytidine analog is guadecitabine, while is still other embodiments, the cytidine analog is 5-azacitidine.

The effective amount of the cytidine analog administered to the patient may vary depending on the cytidine analog being used, the size and age of the patient, and the disease being treated. In general, the effective amount of the cytidine analog (e.g., decitabine) will be between 5 and 100 mg/m², such as about 10-95, about 15-85, or about 20-75 mg/m². In some embodiments, the effective amount may be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² and this amount may be administered once daily, once every other day, once every 3 days, once every 4 days, once every 5 days, once every 6 days, or once a week. The effective amount of the cytidine analog (e.g., decitabine) may be administered to the patient at least about 1 day, at least about 2 days, at least about 3 days, at least about 4 days, at least about 5 days, at least about 6 days, at least about a week, at least about 8 days, at least about 9 days, at least about 10 days, at least about 11 days, at least about 12 days, at least about 13 days, or at least about 2 weeks prior to administration of the CD123-targeted therapy.

### IV. Pharmaceutical Compositions and Formulations

Pharmaceutical compositions suitable for use in the methods described herein can include the CD123-targeted therapy (e.g., an anti-CD123 CAR or ant-CD123 antibody) and a pharmaceutically acceptable carrier or diluent, as well as a cytidine analog (e.g., decitabine, guadecitabine, or 5-azacytidine) and a pharmaceutically acceptable carrier or diluent.

The pharmaceutical compositions may be formulated for intravenous, subcutaneous, intraperitoneal, intramuscular, oral, nasal, pulmonary, ocular, vaginal, or rectal administration. In some embodiments, a CD123-targeted therapy and/or the cytidine analog can be formulated for intravenous, subcutaneous, intraperitoneal, or intramuscular administration, such as in a solution, suspension, emulsion, liposome formulation, etc. The pharmaceutical compositions can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Pharmaceutical compositions of the disclosure can be administered in combination with other therapeutics. For example, the combination therapy can include a pharmaceutical composition comprising at least one of the disclosed CD123-targeted therapies in combination with at least one or more additional therapeutic agents, including but not limited to, CAR T cells directed to another molecular target (e.g., modified T cells that express an anti-CD19, anti-Her2, anti-CS-1, anti-PSCA, anti-IL13R, or anti-CD20 CAR), other tumor-targeting antibodies (e.g., an anti-CAIX antibody or an anti-PD-L1 antibody), immune response potentiating modalities (e.g., an anti-GITR antibody, an anti-OX40 antibody, an anti-CD137 antibody, or a TLR agonist), and small molecule drugs (e.g., venetoclax, a BTK inhibitor, an EGFR inhibitor, a BET inhibitor, a PI3Kdelta inhibitor, a BRAF inhibitor, or a PARP inhibitor). The pharmaceutical compositions of the disclosure can also be administered in conjunction with radiation therapy.

### V. Methods of Treating Hematological Cancer

Provided herein are methods of treating hematological cancer, malignant disease, or cancer cell proliferation with the disclosed combination of a cytidine analog (e.g., decitabine) and a CD123-targeted therapy. More specifically, the disclosure provides for methods of upregulating CD123 expression on cancer cells by administering a pre-treatment comprising an effective amount of a cytidine analog (e.g., decitabine) to a subject with a hematological cancer, and then subsequently administering to that patient a CD123-targeted therapy (e.g., an anti-CD123 CAR, an anti-CD123 antibody, etc.).

Blastic plasmacytoid dendritic cell neoplasm (BPDCN) is a rare, clinically aggressive hematologic malignancy derived from the precursors of plasmacytoid dendritic cells. It can also affect lymph nodes, liver, spleen, skin and other extramedullary sites. The disease invariably progresses and results in leukemic blasts involving the bone marrow and peripheral blood. There have been a variety of therapeutic strategies utilized for the treatment of either frontline or relapsed/refractory BPDCN, but currently there are no approved therapies. Most groups have implemented multi-agent chemotherapy regimens inspired by AML, acute lymphoblastic leukemia (ALL), or lymphoma treatment regimens. However, despite these intensive therapies, median overall survival for adults with BPDCN remains approximately 8-14 months. Younger, fit patients may do better if eligible for stem cell transplant, but the majority of BPDCN patients are older with multiple co-morbidities so are not eligible for transplant.

Acute myeloid leukemia (AML) is a biologically heterogeneous group of related diseases of bone marrow and blood. AML has the highest death rate of all leukemias. The majority of patients who achieve complete remission/response (CR) after standard therapy will relapse, many within one year, unless they receive allogeneic stem cell transplantation (alloSCT). Others might not even be able to achieve CR after first line induction therapy. The 5-year overall survival (OS) from first relapse for these patients is only 10%.

Myelodysplastic syndromes (MDSs) are a group of myeloid neoplasms characterized by peripheral blood cytopenias, including RBC-transfusion dependence, and increased risk of leukemic evolution. MDSs range from indolent conditions with a long natural history to high-risk subtypes with outcomes analogous to AML. The only potentially curative treatment of patients with high-risk MDS (hrMDS) is alloSCT. The median overall survival for hrMDS patients is only 8.4-18 months.

Thus, for patients with BPDCN, AML and hrMDS, there is an urgent unmet need for the development of safe and effective therapies.

CD123, the alpha-subunit of the heterodimeric interleukin-3 receptor (IL-3R), is expressed on the surface of AML blasts, residual leukemic cells (RLCs) and leukemic stem cells (LSCs). Eighty to ninety-three percent of AML samples tested express CD123. Uniformly high levels of CD123 expression is a histopathologic hallmark for BPDCN. In MDS, >50% of patients express CD123, and hrMDS patients more frequently express CD123. Because CD123 is a distinguishing marker of AML stem cells, BPDCN and MDS cells, it may be used to selectively and therapeutically to target these chemo-resistant, malignant cells. Furthermore, it has been reported that decitabine (and potentially other cytidine analogs) can upregulate CD123 expression in AML blasts in vitro. Accordingly, the addition of decitabine to a conditioning or pre-treatment regimen of patients with BPDCN, AML, or MDS will increase the chance that CD123-targeted therapies (e.g., CD123-specific CAR T-cells) will recognize their leukemic blast target cells.

In some embodiments, the disclosed method of treating a hematological cancer comprises administering an effective amount of a cytidine analog to an individual with a hematological cancer and subsequently administering to the individual a therapeutic agent that targets CD123. In some embodiments, the cytidine analog is selected from the group consisting of decitabine, guadecitabine, and 5-azacytidine. In some embodiments, the cytidine analog is decitabine and the therapeutic agent that targets CD123 is a T-cell or NK cell that expresses a CD123-specific. In particular embodiments, the CD123-specific CAR comprises a CD123 binding domain comprising SEQ ID NOs: 1/2 or 3/4, a hinge (e.g., an IgG4 hinge or derivative thereof) a transmembrane domain (e.g., CD8 or CD28), a costimulatory domain (e.g., 4-1BB or CD28), and a CD3t intracellular signaling domain. In some embodiments, the effective dose of the cytidine analog (e.g., decitabine) may be about 5 to about 100 mg/m² (e.g., about 20 mg/m²), and this dose may be administered daily for about 3 to about 5 days starting at least a week prior to the administration of the cells expressing the CD123-specific CAR.

In some embodiments, the therapeutic agent that targets CD123 may be a monoclonal antibody (e.g., talacotuzumab), a bispecific antibody (e.g., flotetuzumab, XmAb14045, JNJ-63709178, APV0436, or APV0437), an antibody-drug conjugate (e.g., SGN-CD123A or IMGN632), and an immunotoxin-peptide conjugate (e.g., SL-401).

In some embodiments, the disclosed method of conditioning an individual with blastic plasmacytoid dendritic cell neoplasm (BPDCN), acute myeloid leukemia (AML), or Myelodysplastic Syndrome (MDS) for treatment with T-cells (or NK cells) expressing a chimeric antigen receptor (CAR) that binds to CD123 (i.e., a CD123-specific CAR) comprises administering to the individual with BPDCN, AML, or MDS an effective dose of decitabine, thereby increasing expression of CD123 on BPDCN, AML or MDS stem cells and/or blast; and administering to the individual a population of T-cells (or NK cells) expressing a CAR that binds to CD123. In some embodiments, the effective dose of decitabine may be about 5 to about 100 mg/m² (e.g., about 20 mg/m²), and this dose may be administered daily for about 3 to about 5 days starting at least a week prior to the administration of the cells expressing the CAR that binds to CD123.

In some embodiments, the disclosed method of conditioning an individual with blastic plasmacytoid dendritic cell neoplasm (BPDCN), acute myeloid leukemia (AML), or Myelodysplastic Syndrome (MDS) for treatment with T-cells expressing a chimeric antigen receptor (CAR) that binds to CD123 comprises administering to the individual an effective dose of decitabine at least about a week prior to administration of a CAR, thereby increasing expression of CD123 on BPDCN, AML, or MDS stem cells and/or blast prior to administration of a CAR; and administering to the individual a population of T-cells expressing a CAR that binds to CD123. In some embodiments, the effective dose of decitabine may be about 5 to about 100 mg/m² (e.g., about 20 mg/m²), and this dose may be administered daily for about 3 to about 5 days starting a week prior to the administration of the cells expressing the CAR that binds to CD123.

Non-limiting examples of hematological cancers include lymphoma, Non-Hodgkin's lymphoma, chronic lymphocyctic leukemia, multiple myeloma, blastic plasmacytoid dendritic cell neoplasm (BPDCN), acute myeloid leukemia (AML), or Myelodysplastic Syndrome (MDS). More specifically, the hematological cancers that are most well-suited for the disclosed methods of treatment include those cancers that express CD123. In some embodiments, the hematological cancer being treated already overexpressed CD123 prior to administration of a cytidine analog (e.g., decitabine), while in some embodiments, the hematological cancer being treated may have little or no CD123 expression prior to administration of a cytidine analog (e.g., decitabine).

Dosage regimens for pre-treatment with a cytidine analog can be adjusted to provide the optimum desired response (e.g., upregulation or increased surface expression of CD123). For example, in some embodiments, pre-treatment with a cytidine analog (e.g., decitabine) may comprise administering an effective amount of the drug to a patient once at least one week prior to administration of the CD123-targeted therapy, once daily for at least one week prior to administration of the CD123-targeted therapy, once every other day for at least one week prior to administration of the CD123-targeted therapy, once every three days for at least one week prior to administration of the CD123-targeted therapy, once at least two weeks prior to administration of the CD123-targeted therapy, once daily for at least two weeks prior to administration of the CD123-targeted therapy, once every other day for at least two weeks prior to administration of the CD123-targeted therapy, once every three days for at least two weeks prior to administration of the CD123-targeted therapy, or once a week for at least two weeks prior to administration of the CD123-targeted therapy. In some embodiments, the patient may be treated with a cytidine analog for at least 3 days, at least 4 days, or at least 5 days during the week prior to administration of a CD123-targeted therapy. For instance, a patient may receive decitabine or azacitadine on days −7, −6, −5, −4, and/or −3 or on days −7, −6, and −5 prior to commencing treatment with the CD123-targeted therapy on day 0.

In some embodiments, the patient may continue to be treated with the cytidine analog (e.g., decitabine) for a time period following initiation of the CD123-targeted therapy, for instance, for at least about a week, at least about 2 weeks, at least about 3 weeks, or for the entire duration of the CD123-targeted therapy treatment regimen.

Similarly, dosing regimens for the CD123-targeted therapy may vary depending on the therapy being administered (e.g., a CD123-specific CAR or anti-CD123 monoclonal antibody), among other factors and the optimum desired response (e.g., a therapeutic response like tumor regression, reduction in malignant cell count, or remission). For instance, the CD123-targeted therapy may be administered as a single bolus may be administered, while in some embodiments, several divided doses may be administered over time or the dose may be proportionally reduced or increased as indicated by the situation. In some embodiments the CD123-targeted therapy may be administered once or twice weekly by subcutaneous or intravenous injection. In some embodiments, the CD123-targeted therapy may be administered once or twice monthly by subcutaneous or intravenous injection. In some embodiments, the CD123-targeted therapy may be administered once every week, once every other week, once every three weeks, once every four weeks, once every other month, once every three months, once every four months, once every five months, or once every six months.

Exemplary doses can vary according to the size and health of the individual being treated, as well as the condition being treated, as discussed in more detail above in the sections relating to the CD123-targeted therapies and cytidine analogs. For example, in some embodiments, decitabine may be administered at about 20 mg/m² per day for days −7 to −3 and a CD123-specific CAR may be administered at about 600×10⁶ cells on day 0.

Particular treatment regimens may be evaluated according to whether it will improve a given patient's outcome, meaning it will reduce the risk of recurrence of the hematological cancer being treated or increase the likelihood of progression-free survival of the given cancer.

Thus, for the purposes of this disclosure, a subject is treated if one or more beneficial or desired results, including desirable clinical results, are obtained. For example, beneficial or desired clinical results include, but are not limited to, one or more of the following: decreasing one or more symptoms resulting from the disease, increasing the quality of life of those suffering from the disease, decreasing the dose of other medications required to treat the disease, delaying the progression of the disease, and/or prolonging survival of the patient.

Furthermore, while the subject of the methods is generally a human cancer patient, the age of the patient is not limited. The disclosed methods are useful for treating cancer, malignant disease, or cancer cell proliferation with various recurrence and prognostic outcomes across all age groups and cohorts. Thus, in some embodiments, the subject may be a paediatric subject, while in other embodiments, the subject may be an adult subject.

The following examples are given to illustrate the present invention. It should be understood, however, that the invention is not to be limited to the specific conditions or details described in these examples.

### EXAMPLES

### Example 1—Phase I/II Clinical Trial Treating BPDCN, AML, and MDS with the Disclosed Combination Therapy

A phases I/II open label, multicenter trial was designed to assess the safety and efficacy of a CD123-specific CAR in patients with relapsed or refractory Blastic Plasmacytoid Dendritic Cell Neoplasm (BPDCN), Acute Myeloid Leukemia (AML), and high risk Myelodysplastic Syndrome (MDS).

### Rationale

Adoptive cellular immunotherapy (ACIT) is a promising treatment approach for a variety of malignancies. Adoptively transferred T cells for the present example were genetically modified using a self-inactivating (SIN) lentiviral vector to express a CD123-specific, CD28-costimulatory CAR with a CD3ζ intracellular signaling domain. The CD123-specific CAR was demonstrated after to have killing activity in treatment of CD123⁺ cell lines and primary AML blasts from patient samples in vitro. Subsequently, these data supported initiation of a Phase I trial.

Two out of six patients with AML (receiving 200 million CAR-T⁺ cells) and 1/1 patients with BPDCN (receiving 100 million CAR-T⁺ cells) achieved complete remission (CR), with durations of 245, 48 and 100 days, respectively. The maximum dose level to be tested will be 500 million CAR-T⁺ cells (6.7×10⁶ cells/kg).

### Study Design

This was a multicenter, Phase 1/2, open-label, nonrandomized trial of a CD123-specific CAR in patients with relapsed/refractory BPDCN, relapsed or refractory AML, or demethylation resistant hrMDS.

In the Phase 1 portion of the trial, three escalating dose levels (DL1, DL2, DL3) were tested using a 3+3 design. The starting dose level chosen (DL1) was one level below the highest safe dose level tested of the CAR-T cells (CD123.CD28.CD3ζ). The highest dose level proposed was 500×10⁶ CAR-T⁺ cells. Projected dose levels to be tested are outlined in the table below.

Dose Levels (×10⁶ CAR-T⁺ cells) (−20%)

| | |
|---|---|
| DL-1 | 50 |
| DL 1 | 100 |
| DL 2 | 300 |
| DL 3 | 600 |

The Phase 2 portion of the trial was divided into three arms to evaluate the efficacy of the CD123-specific CAR in newly diagnosed and relapsed BPDCN (Arm 1), relapsed/refractory de novo and secondary AML (Arm 2) and demethylation resistant high-risk MDS (Arm 3).

Arms 1-3 of Phase 2 had an interim efficacy analysis for futility prior to completion of recruitment to the arm. Safety was also analyzed at this interim point in all three arms by the Data Safety Monitoring Board (DSMB).

### Study Endpoints

Phase 1—Primary endpoints include:

To assess the safety and tolerability of the CAR T-cells infusion in patients with relapsed or refractory BPDCN, AML and hrMDS; and

To determine the recommended Phase 2 dose of the CAR T-cells in patients with relapsed or refractory BPDCN, AML, and hrMDS.

Phase 2—Primary endpoints include:

Blastic Plasmacytoid Dendritic Cell Neoplasm

To assess the efficacy of the CAR T-cells infusion in relapsed or refractory BPDCN patients as measured by Response Rate which consists of Complete Remission and Clinical Complete Remission and Complete Remission with incomplete hematologic recovery (CR+CRc+CRi) at day 28 post infusion

Acute Myeloid Leukemia

To assess the efficacy of MB-102 infusion in patients with relapsed or refractory AML as measured by Response Rate which consists of Complete Remission and Complete Remission with incomplete hematologic recovery (CR+CRi) at day 28 post infusion

High Risk Myelodysplastic Syndrome

To assess the efficacy of MB-102 infusion in patients with demethylation resistant high risk MDS as measured by Response Rate which consists of Complete Remission, Partial Remission and marrow Complete Remission (CR+PR+mCR) at day 28 post infusion

For patients that have not had an adequate marrow recovery at day 28, the primary efficacy assessment will be analyzed at an appropriate timepoint, as per investigator discretion up to day 84.

Secondary Endpoints include:
To assess the efficacy of MB-102 infusion in BPDCN patients as measured by:
a. Duration of Response (DoR)
b. Progression-Free Survival (PFS)
c. overall survival (OS)
d. CR^{MRD-}Response Rate
To assess the efficacy of MB-102 infusion in patients with relapsed/refractory AML as measured by:
a. DoR
b. Event-Free Survival (EFS)
c. Relapse-Free Survival (RFS)
d. OS
e. CO^{MRD-} Response Rate
To assess the efficacy of MB-102 infusion with high risk MDS as measured by:
a. Hematologic Improvement (HI),
b. Clinical benefit rate (CR+PR+HI+marrow CR)
c. Rate of cytogenetic CR
d. DoR
e. Rate of leukemic transformation
f. EFS
g. PFS
h. OS
i. Transfusion independence
j. CO^{MRD-} Response Rate
Quality of Life (QoL) as measured by European Organization for Research and Treatment of Cancer (EORTC) QLQ-C30 version 3.0, the Functional Assessment of Cancer Therapy-Bone Marrow Transplant (FACT-BMT) version 4.0 and the Functional Assessment of Cancer Therapy-Leukemia (FACT-Leu) version 4.0
To confirm the absence of replication competent lentivirus

Exploratory Endpoints include:
Expansion, trafficking and persistence of the CAR T-cells in blood, bone marrow and other sites of disease, if applicable;
Phenotypic and functional characterization of the CAR T-cells cells both at time of infusion and at different time-points post-infusion;
Cytokine and C-Reactive Protein (CRP) levels;
Relationship between baseline level of CD123 expression and clinical outcome;
Genetic characterization of blast cells pre and post MB-102 treatment; and
immune response to the CAR T-cells.

Approximately 126 were enrolled in the trial including 26 with BPDCN, 41 with AML, and 41 with MDS.

### Dosing and Treatment Schedule

Patients received a pre-treatment regimen of decitabine at 20 mg/m² per day for days −7 to −3.

A lymphodepletion regimen ran from days −5 to −3 in which the patients received Fludarabine 30 mg/m²/day IV (3 days) on days −5, −4, and −3; as well as Cyclophosphamide at 300-500 mg/m²/day IV (3 days) on days −5, −4, and −3.

Treatment with the CD123-specific CAR was commenced on Day 0 by administering 600×10⁶ CAR T-cells.

### Example 2—Determination of Hypomethylating Agents on CD123 Expression

The present example relates to the evaluation of the effects of hyomethylating agents decitabine, 5-azacytidine, and guadecitabine on CD123 expression in various AML cell lines including Kasumi-1 (CD123 low), SKM-1 (CD123 medium), MOLM13 (CD123 high), and primary CD34+enriched bone marrow from healthy donors. Additionally, the expression of PDL-1 (Programmed death-ligand 1)/CD274, a 40kDa type 1 transmembrane protein that plays a major role in suppressing the immune response was also monitored. The scope of this study was to assess the impact of Decitabine as a pre-conditioning regimen for the CD123-specific CAR used in the Phase I clinical trial detailed in Example 1 by addressing potential on-target, off-tumor toxicity.

SKM1, Kasumi-1 and MOLM-13 cell lines were maintained in 80% RPMI 1640+20% FBS according to manufacturer's instructions. Logarithmically growing SKM1, Kasumi-1 and MOLM-13 cell lines were plated at 7,500 cells per well in 384 well microtiter plates and treated with drugs in triplicate using an acoustic liquid handler using protocol (P.SOP.007.02_Compound Addition Using GBG).

The following compounds were serially dosed in 24-hour intervals using an acoustic liquid handler. Decitabine, 5-azacytidine and guadecitabine were dosed at 3000, 1000, 333.33, 111.11, 37.04, 12.35, 4.12, 1.37 and 0.46 nM at 0, 24 and 48 hours. In addition, guadecitabine was dosed at 3000, 1000, 333.33, 111.11, 37.04, 12.35, 4.12, 1.37 and 0.46 nM at 0 hours without serial dosing (single dose). The dose range of the compounds was determined by back calculating the clinical dose and corresponding to a plasma concentration at 1.5 hours after standard dosing. The 1000 nM concentration of decitabine is based on the clinical dose of 20 mg/m², 1-hour intravenous infusion, one time per day for five consecutive days every 4 weeks and the maximum plasma concentration after standard dosing (1.15 micromolar). Decitabine doses in the study were selected above and below this dose. The 1000 nM concentration of 5-azacytidine is based on the clinical dose of 75 mg/m²/day for 7 days every 28 days and the plasma concentration at 1.5 hours after standard dosing. 5-Azacytidine doses in the study were selected above and below this dose. Guadecitabine regimen is 60 mg/m² given subcutaneously daily on Days 1-5 in 28-day cycles (delayed as needed to allow blood count recovery). The clinically relevant guadecitabine dose is 211 nM. Cells were stained at day 0, day 3 and day 7 following cell plating samples were stained with the antibody panel details below and readout with an lntellicyt iQue Plus flow cytometer.

Cells were stained using Notable labs no wash protocol number (P.SOP.009.01_Screen Readout Automated), briefly, antibodies are added to cells with an acoustic liquid handler, incubated at 4° C. for 20 minutes and readout with an Intellicyt iQue Plus flow cytometer. Staining Panel for Cell lines: DAPI, CD123, PDL1, CD34, CD33, CD15, CD38. Live cells were gated using FSC/SSC and DAPI exclusion using FlowJo analysis software (BeckmanFLow), and then further defined by cell surface marker expression. Absolute counts or Mean Fluorescence Intensity from triplicate wells were averaged and fold change was calculated with respect to a vehicle-only (DMSO) control.

CD34 enriched bone marrow was purchase from AllCells. Fresh (never frozen) cells were plated at 15,000 cells per well in 384 well microtiter plates and treated with drugs in triplicate using an acoustic liquid handler. The following compounds were dosed serial in 24-hour intervals: decitabine, guadecitabine and 5-azacytidine were dosed at 3000, 1000, 333.33, 111.11, 37.04, 12.35, 4.12, 1.37 and 0.46 nM at 0, 24 and 48 hours. In addition, auadecitabine was dosed at 3000, 1000, 333.33, 111.11, 37.04, 12.35, 4.12, 1.37 and 0.46 nM at 0 hours without serial dosing (single dose). Cells were stained at day 0 and day 7 following cell plating samples were stained with the antibody panel details below and readout with an Intellicyt iQue Plus flow cytometer. Cells were stained using Notable labs staining protocol number (P.SOP.009.01_Screen Readout Automated). Staining Panel for Healthy CD34+ Bone Marrow: DAPI, CD45, CD3, CD19, CD16, CD14, CD38, CD33, CD34, CD90, CD123, CD274. Live cells were gated using FSC/SSC and DAPI exclusion using FlowJo analysis software (Beckman Flow), and then further defined by cell surface marker expression. Absolute counts from triplicate wells were averaged and normalized to a vehicle-only (DMSO) control.

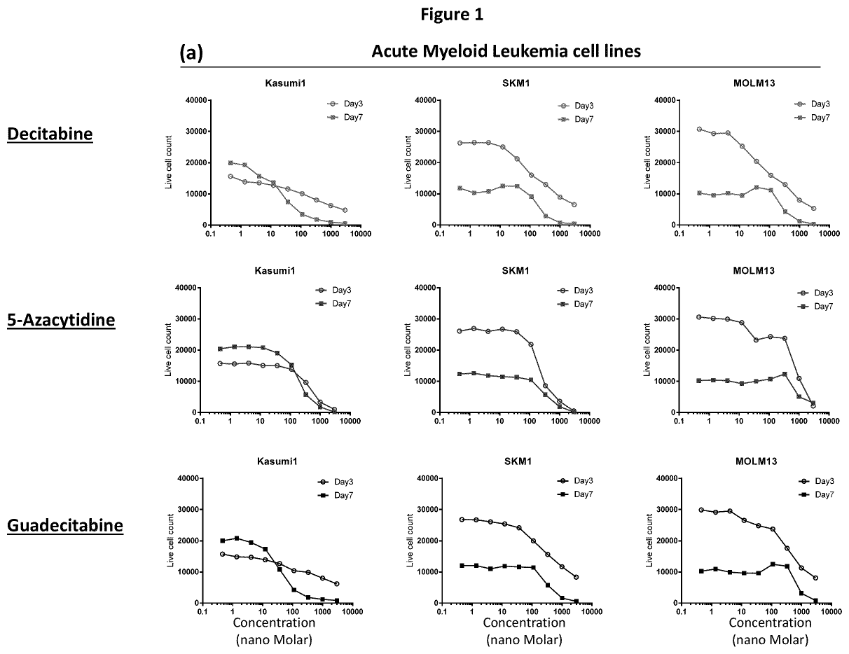

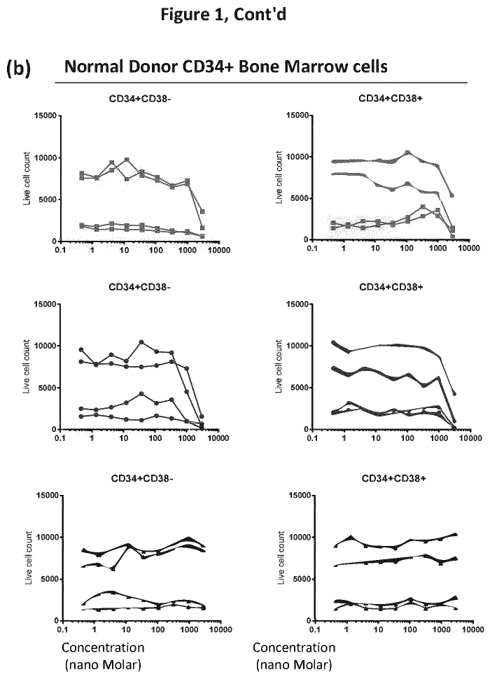

The impact of decitabine, 5-Azacytidine and guadecitabine on cell viability was assessed by flow cytometry. Positive control for cell death, the Staurosporine treatment group, had significant reductions in viable cells in all cell line and primary cell groups. Treatment with all three drugs showed a dose dependent decline in cell numbers for the AML cell lines on day 3. Enhanced cell death was observed on day 7 post treatment in all groups (FIG. 1a). Primary cells CD34+CD38+ Multipotent progenitors (MMPs) and CD34+CD38− Hematopoietic Stem Cells (HSCs) showed a dose dependent decline in cell counts in response to decitabine and 5-azacytidine but not guadecitabine (FIG. 1b).

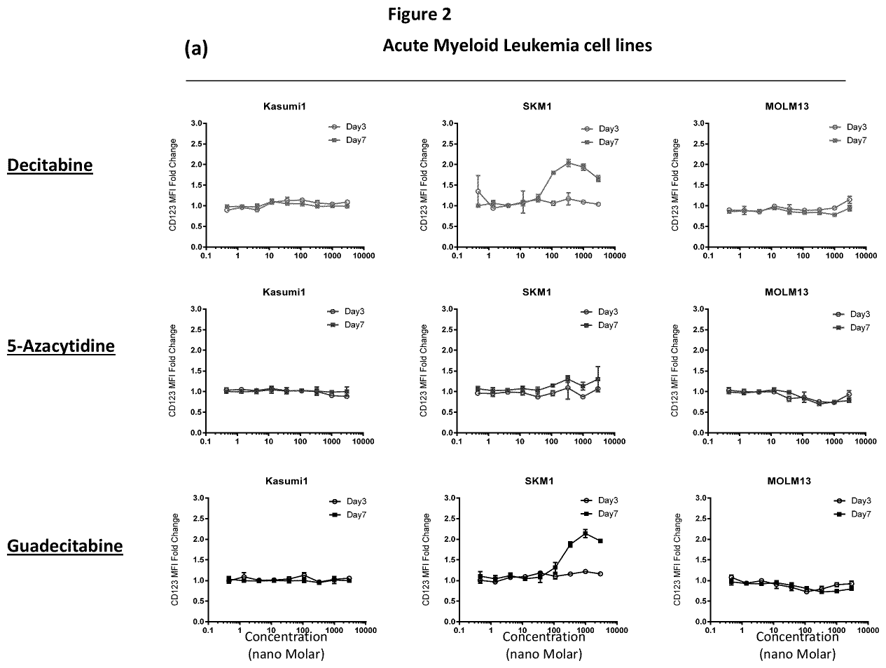

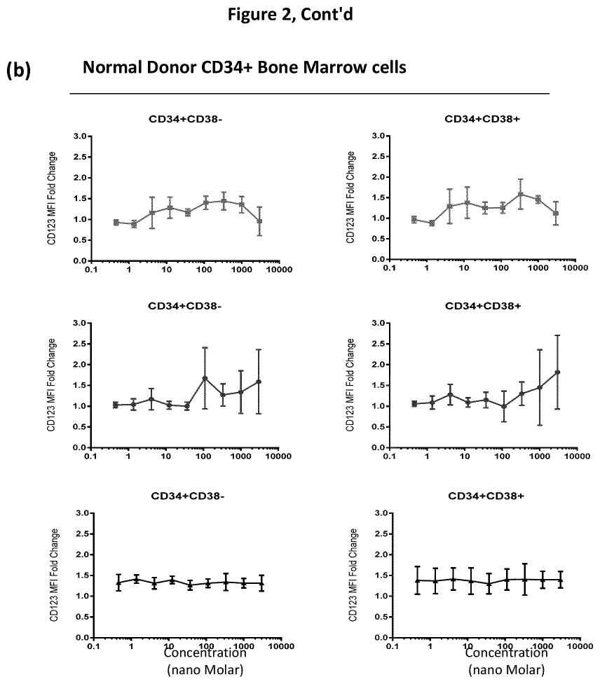

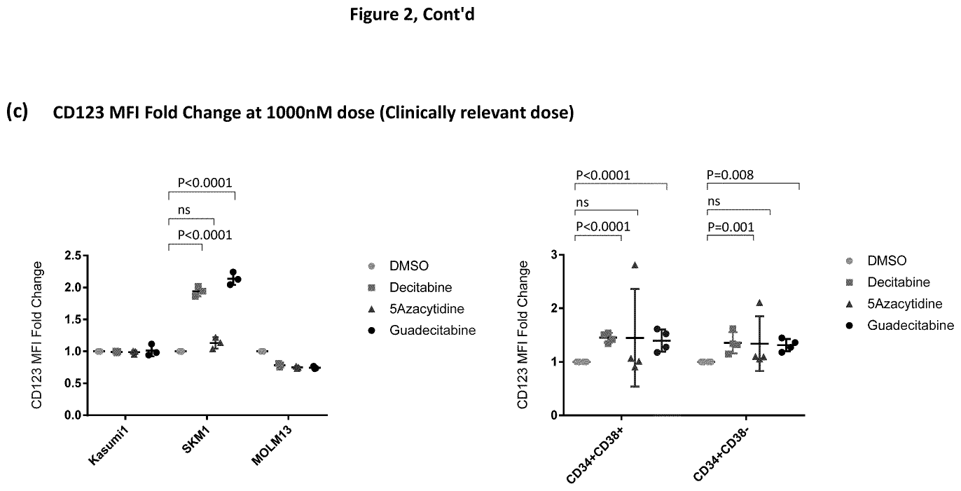

Next, the impact of decitabine, 5-azacytidine and guadecitabine on CD123 expression was evaluated. Decitabine treatment showed a dose dependent increase in CD123 MFI at day 7 in SKM1 cell line but not in Kasumi-1 and MOLM-13. There was 1.3, 1.6- and 1.4-fold increase in CD123 expression at 111.11, 333.33 and 1000nM dose respectively in CD38+CD34-Hematopoietic stem cells. The CD38+CD34+ MMPs showed an average increase of 1.4, 1.4 and 1.3-fold in CD 123 expression at 111.11, 333.33 and 1000 nM dose. Cells treated with High dose of decitabine at 3000 nM showed cell death (FIG. 1b) and no change in MFI with respect to DMSO treated cells (FIG. 2b). In contrast, 5-Azacytidine treatment did not impact CD123 MFI in all three cell lines tested (FIG. 2b, middle panel). Additionally, CD123 MFI was highly variable amongst the four donors tested. Like decitabine, guadecitabine showed increase in CD123 MFI in a dose dependent manner for SKM1 cell line but not Kasumi-1 or MOLM13. Guadecitabine treatment did not show any change in CD123 expression in normal bone marrow HSCs or MMPs regardless of the dose administered. FIG. 2c shows both decitabine and guadecitabine significantly enhanced CD123 expression in SK1, HSC and MMPs at 1000 nM clinically relevant dose.

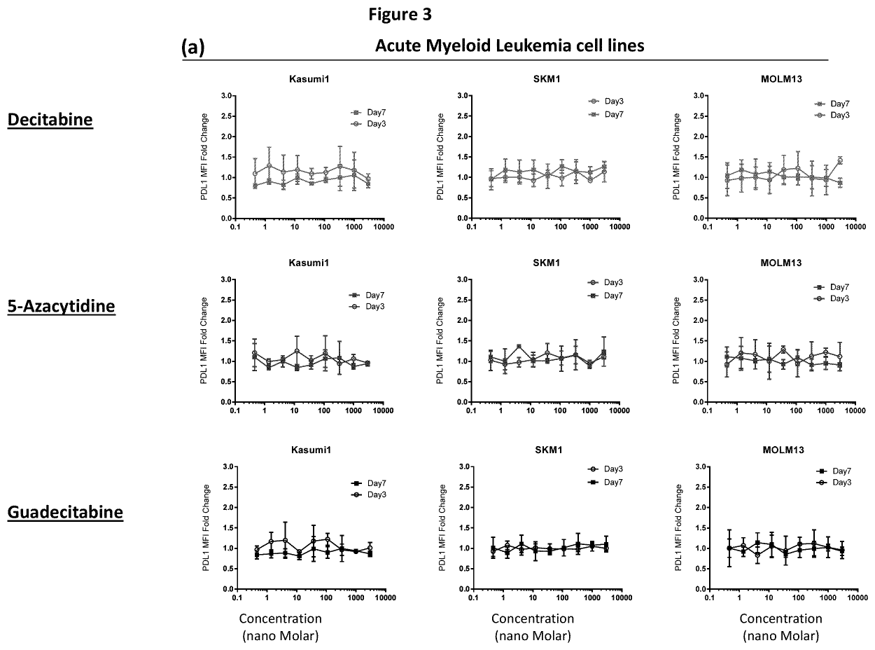

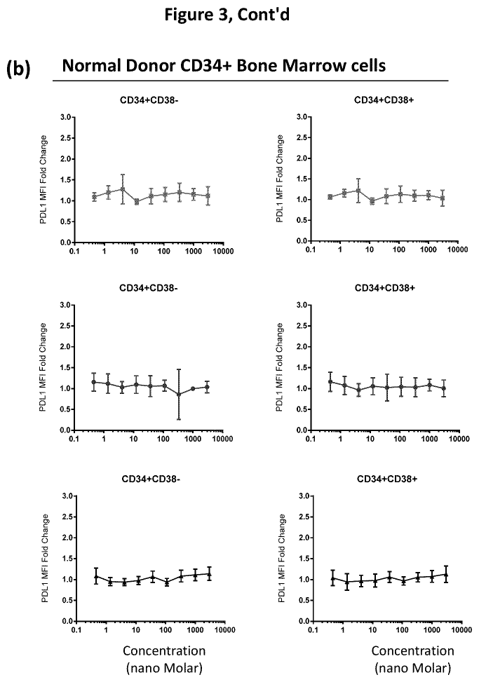

Impact of decitabine, 5-azacytidine and guadecitabine on PD-L 1 expression was evaluated. As shown in FIG. 3 no impact on PD-L 1 was observed post decitabine, 5-azaycytidine or guadecitabine treatment at any of the dose for all cell lines and primary bone marrow HPCs or MMPs.

Decitabine showed an increase in CD123 expression for SKM-1 cells but not MOLM13 and Kasumi-1. Next, decitabine exhibited an increase in CD123 MFI in primary CD34+ Bone marrow cells at a clinically relevant dose of 1000 nM. PD-L1 expression was not impacted by any of the drugs tested, suggesting decitabine does not upregulate inhibitory receptors that may impair CD123 CAR-T function.

### Example 3—Clinical Experience using Hypomethylating Agent as Part of Lymphodepletion Regimen

Fifteen AML patients treated in our ongoing first in human phase 1 clinical trial (NCT0262355) evaluating CD123CAR T cells in patients with relapsed or refractory AML or blastic plasmacytoid dendritic cell neoplasm (BPDCN) received lymphodepletion prior to CAR T cell infusion on day 0. All received fludarabine (25-30 mg/m2/day) and cyclophosphamide (300-500 mg/m2/day). Both were administered daily from days −5 to −3. Eight also received decitabine (20 mg/m2/day) given daily for 5 days from days −7 to −3 or daily for 3 days from days −5 to −3. All patients tolerated lymphodepletion and CD123CAR T cell treatment well with no dose limiting toxicities. We have not observed increased treatment related adverse events in the decitabine treated group. Due to the limited number of treated patients (8 patients treated with decitabine and 7 treated without decitabine), statistical difference of incidences of treatment related adverse events and efficacy between these 2 groups cannot be calculated. Nevertheless, this clinical experience suggests that lymphodepletion regimen containing decitabine, fludarabine and cyclophosphamide is a feasible and safe regimen.

Based on the in vitro and the clinical data provided in these examples, it can be concluded that decitabine upregulates CD123 expression in select AML cell line SKM1 and primary bone marrow cells. However, the increase in CD123 is not sufficient to cause adverse off-tumor effects as evident by our clinical data which demonstrates, pre-treatment with decitabine followed by CD123 CAR-Twas well tolerated. The clinical observation may be a result of higher activation threshold required by CD123 CAR-T to mount an anti-tumor response.

Taken together, decitabine treatment may upregulate CD123 expression in select AML cell lines and at select doses in bone marrow cells in vitro. As our clinical data did not present any potential safety risks, this justifies the use of decitabine in a pre-treatment conditioning regimen prior to administration of a CD123-targeted therapy.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

1. A method of treating a hematological cancer comprising, administering an effective amount of a cytidine analog to an individual with a hematological cancer and subsequently administering to the individual a therapeutic agent that targets CD123.

2. The method of claim 1, wherein the cytidine analog is selected from the group consisting of decitabine, guadecitabine, and 5-azacytidine.

3. The method of claim 1, wherein the cytidine analog is decitabine.

4. The method of claim 1, wherein the hematological cancer is blastic plasmacytoid dendritic cell neoplasm (BPDCN), acute myeloid leukemia (AML), or Myelodysplastic Syndrome (MDS).

5. The method of claim 1, wherein the hematological cancer is characterized by cancerous cells that overexpress CD123.

6. The method of claim 1, wherein the therapeutic agent is a T-cell or a natural killer (NK) cell expressing a chimeric antigen receptor (CAR) that binds to CD123.

7. The method of claim 6, wherein the CAR comprises (i) the complementarity determining regions (CDRs) of the heavy chain variable region disclosed in SEQ ID NO:1 and the CDRS of the light chain variable region disclosed in SEQ ID NO:2; or (ii) the CDRs of the heavy chain variable region disclosed in SEQ ID NO:3 and the CDRs of the light chain variable region disclosed in SEQ ID NO:4.

8. The method of claim 6, wherein the CAR comprises (i) a heavy chain variable region comprising SEQ ID NO:1 and a light chain variable region comprising SEQ ID NO:2; or (ii) a heavy chain variable region comprising SEQ ID NO:3 and a light chain variable region comprising SEQ ID NO:4.

9. The method of claim 7, wherein the CAR comprises a CD28 costimulatory domain, a 4-1BB costimulatory domain, or a combination thereof; and wherein the CAR comprises a CD28 transmembrane domain, a CD4 transmembrane domain, or a CD8 transmembrane domain; a hinge domain derived from an IgG4 Fc region or an IgG2 Fc region.

10. (canceled)

11. (canceled)

12. The method of claim 7, wherein the CAR comprises a CD123 binding domain, a CD28 costimulatory domain, a CD28 transmembrane domain, a hinge derived from an IgG4 Fc region, and a CD3 domain.

13. The method of claim 7, wherein the CAR comprises SEQ ID NO:5 or SEQ ID NO:6.

14. The method of claim 6, wherein the CAR is bispecific for CD123 and a different antigenic target.

15. (canceled)

16. The method of claim 1, wherein the therapeutic agent is selected from the group consisting of a monoclonal antibody, a bispecific antibody, an antibody-drug conjugate, and an immunotoxin-peptide conjugate.

17. The method of claim 16, wherein the antibody is talacotuzumab; wherein the antibody-drug conjugate is SGN-CD123A or IMGN632; or wherein the immunotoxin-peptide conjugate is SL-401.

18. (canceled)

19. (canceled)

20. (canceled)

21. The method of claim 1, wherein the decitabine is administered at a dose of about 20 mg/m² per day.

22. The method of claim 1, wherein the decitabine is administered to the subject at least one week prior to treatment with the therapeutic agent.

23. A method of conditioning an individual with blastic plasmacytoid dendritic cell neoplasm (BPDCN), acute myeloid leukemia (AML), or Myelodysplastic Syndrome (MDS) for treatment with T-cells expressing a chimeric antigen receptor (CAR) that binds to CD123 comprising,
a. administering to the individual with BPDCN, AML, or MDS an effective dose of decitabine, thereby increasing expression of CD123 on BPDCN, AML or MDS stem cells and/or blast; and
b. administering to the individual a population of T-cells expressing a CAR that binds to CD123.

24. The method of claim 23, wherein the effective dose of decitabine comprises a dose of about 20 mg/m² per day.

25. (canceled)

26. (canceled)

27. (canceled)

28. The method of claim 23, wherein the CAR comprises (i) the complementarity determining regions (CDRs) of the heavy chain variable region disclosed in SEQ ID NO:1 and the CDRS of the light chain variable region disclosed in SEQ ID NO:2; or (ii) the CDRs of the heavy chain variable region disclosed in SEQ ID NO:3 and the CDRs of the light chain variable region disclosed in SEQ ID NO:4.

29. The method of claim 23, wherein the CAR comprises (i) a heavy chain variable region comprising SEQ ID NO:1 and a light chain variable region comprising SEQ ID NO:2; or (ii) a heavy chain variable region comprising SEQ ID NO:3 and a light chain variable region comprising SEQ ID NO:4.

30. The method of claim 23, wherein the CAR comprises a CD123 binding domain, a CD28 costimulatory domain, a CD28 transmembrane domain, a spacer derived from an IgG4 Fc region, and a CD3ζ domain.

31. The method of claim 23, wherein the CAR comprises SEQ ID NO:5 or SEQ ID NO:6.

32. The method of claim 23, wherein the CAR is bispecific for CD123 and a different antigenic target.

33. (canceled)

34. The method of claim 23, wherein the population of T-cells expressing a CAR that binds to CD123 is administered at a dose of 1.0×10⁴-12.0×10⁶ cells/kg.

35. The method of claim 23, wherein the decitabine is administered to the subject at least one week prior to treatment with the population of T-cells expressing a CAR that binds to CD123.

36. A method of conditioning an individual with blastic plasmacytoid dendritic cell neoplasm (BPDCN), acute myeloid leukemia (AML), or Myelodysplastic Syndrome (MDS) for treatment with T-cells expressing a chimeric antigen receptor (CAR) that binds to CD123 comprising,
a. administering to the individual an effective dose of decitabine at least about a week prior to administration of a CAR, thereby increasing expression of CD123 on BPDCN, AML, or MDS stem cells and/or blast prior to administration of a CAR; and
b. administering to the individual a population of T-cells expressing a CAR that binds to CD123.

37. (canceled)

38. (canceled)

39. (canceled)

40. The method of claim 36, wherein the CAR comprises (i) the complementarity determining regions (CDRs) of the heavy chain variable region disclosed in SEQ ID NO:1 and the CDRS of the light chain variable region disclosed in SEQ ID NO:2; or (ii) the CDRs of the heavy chain variable region disclosed in SEQ ID NO:3 and the CDRs of the light chain variable region disclosed in SEQ ID NO:4.

41. The method of claim 36, wherein the CAR comprises (i) a heavy chain variable region comprising SEQ ID NO:1 and a light chain variable region comprising SEQ ID NO:2; or (ii) a heavy chain variable region comprising SEQ ID NO:3 and a light chain variable region comprising SEQ ID NO:4.

42. The method of claim 36, wherein the CAR comprises a CD123 binding domain, a CD28 costimulatory domain, a CD28 transmembrane domain, a spacer derived from an IgG4 Fc region, and a CD3 domain.

43. The method of claim 36, wherein the CAR comprises SEQ ID NO:5 or SEQ ID NO:6.

44. The method of claim 36, wherein the CAR is bispecific for CD123 and a different antigenic target.

45. (canceled)

46. The method of claim 36, wherein the population of T-cells expressing a CAR that binds to CD123 is administered at a dose of 1.0-12.0×10⁶ cells/kg.

47. The method of claim 36, wherein the decitabine is administered at a dose of about 20 mg/m² per day.

48. A method of treating a patient diagnosed with a disease characterized by an overproduction of immature blood cells, comprising administering to a patient in need thereof and who has previously received an effective amount of decitabine for at least a week an effective amount of a CD123-targeting therapeutic agent.

49. The method of claim 48 in which the patient continues to receive decitabine after initiation of CD123-targeting therapy for at least an additional 1-2 weeks.

50. The method of claim 49 in which the patient continues to receive decitabine after initiation of CD123-targeting therapy for at least the duration of the CD123-targeting therapy.
